(19) United States
(12) Patent Application Publication
(10) Pub. No.: US 2008/0318262 A1
(43) Pub. Date: Dec. 25, 2008

(54) Protein Cleavage at Aspartic Acid Using Chemical Reagents

(75) Inventors: Joseph Kwon, Kyungbuk (KR); Taehoon Lee, Kyungbuk (KR)

Correspondence Address: LEXYOUME IP GROUP, LLC, 5180 PARKSTONE DRIVE, SUITE 175, CHANTILLY, VA, 20151, US

(73) Assignee: Sigmol, Inc., Pohang (KR)

(21) Appl. No.: 11/575,089

(22) PCT Filed: Sep. 14, 2005
(86) PCT No.: PCT/KR2005/003042

§ 371 Date: Mar. 12, 2007

Related U.S. Application Data
(60) Provisional application No. 60/610,306, filed on Sep. 15, 2004.

(51) Int. Cl. G01N 33/68; C12Q 1/48

(52) U.S. Cl. 435/15; 436/89

(57) ABSTRACT

The present invention relates to the methods of identifying and quantifying polypeptides in a given sample by mass spectrometric analysis. More specifically, the invention provides the methods for sample preparation for proteomic analysis: the methods for the fragmentation of proteins into peptides with the specific cleavage rule (cleavage at amino-terminal or carboxyl-terminal of aspartic acid), which are suitable for the analysis by mass spectrometry apparatus.

### CROSS REFERENCE TO RELATED APPLICATION

The present application claims priority to and the benefit of U.S. provisional application No. 60/610,306 filed in the United State Patent and Trademark Office on Sep. 15, 2004, the entire content of which is incorporated hereinto by reference.

### FIELD OF INVENTION

The present invention provides a method of processing proteins for identification and quantification. To be specific, the invention relates to a kit and an apparatus for processing proteins into peptides by using chemical reagents, and to the methods of using them in broad range of proteomics researches.

### BACKGROUND OF THE INVENTION

Although complete genomic sequencing can provide useful information for the prediction of genes in a given species, the sequences alone do not explain the mechanisms underlying biological and pathophysiological processes, because neither the quantity nor the molecular details of the translated protein product such as structure, functional activity, state of post-translational modification can be precisely predicted. The new discipline of proteomics aims to unravel biochemical information at the molecular level. Therefore, the understanding of the proteome in a given context is essential to assess the physiological state of a cell or organism.

At present, a number of techniques have been developed to address the growing need to identify proteins more quickly and accurately through mass-accurate methods such as mass spectrometry. The platforms used for proteomic analysis involve the integration of two broad practices, including separation and identification of proteins in a sample. Two-dimensional gel electrophoresis (2-DE) or liquid chromatography (LC) of one or more types are used for the separation of proteins prior to mass spectrometry. In 2-DE, the proteins placed in a gel migrate depending largely on molecular weight and isoelectric point, thus generating a characteristic gel pattern. Mass spectrometry is generally used for the identification of proteins. In mass spectrometry, proteins or peptides are ionized and ionized species are subject to electric and/or magnetic fields in a vacuum. Their molecular weights can be deduced from the travel path of the ions. The identities of proteins or peptides can be disclosed by a peptide fingerprinting method or de novo sequencing using mass spectrometry (MS) or tandem MS (MS/MS).

In a widely used strategy for sample preparation in proteomic research, the proteins are enzymatically cleaved into their constituent peptides prior to MS analysis to enhance the likelihood that at least some of the protein is sufficiently ionized so as to be detected by generating the peptides compatible for an accurate analysis in general MS apparatus which has a limited ranges of mass measurement. Peptide mass mapping of proteins separated by polyacrylamide gel electrophoresis with enzymatic digestion has been a routine procedure for protein characterization. The most frequently used protease is trypsin because of its well-defined specificity and the appropriate size of tryptic peptides for mass spectrometric analysis. A few other commercially available proteases such as Lys-C, Glu-C, and Asp-N are discriminated against certain substrates. Having a lot of strong points, tryptic digestion is not a method of choice for hydrophobic or very basic proteins. In addition, protease autolysis products (for example, trypsin; 261.14, 514.32, 841.50, 905.50,1005.48, 1044.56, 1468.72, 1735.84, 1767.79, 2157.02, 2210.10, 2282.17, 3012.32, 4474.09, 4488.11 Da) sometimes interfere with spectrum interpretation. Furthermore, some buffers required for efficient and specific proteolysis may generate chemical noises, thus requiring additional purification before mass spectrometric analysis.

As an alternative to enzymatic digestion, new attempts of protein analysis that use acid hydrolysis have recently been made. Several different approaches have been reported that pertain to efficient cleavage of protein. Bark et al. developed a high-temperature proteolytic digestion method using thermolysin. Gobom et al. suggested vapor-phase acid hydrolysis using pentafluoro propionic acid (PFPA), but three different types of cleavages, including sequence ladder, were observed, thus leading to the unwanted drawbacks of increased spectrum complexity. Aiqun Li at al. suggested chemical cleavage at aspartic acid with 2 (v/v) % formic acid, but unpredictable formylated fragments, which make it very difficult to identify the proteins using peptide-mass fingerprinting (PMF), were generated. Moreover, the formic acid method does not show clear cleavage rule with sequencing data.

In other points of view, various analytical approaches have been reported that utilize bottom-up proteomics and stable isotope labeling to perform relative quantification of proteins. These methods can be broadly classified as either (1) metabolic, where the isotope label is incorporated during protein synthesis, (2) amino acid-specific, where the stable isotope label is applied only to peptides containing a specific amino acid, such as cysteine in the case of the ICAT™ method, and (3) global labeling methods where the label is applied to every peptide in a given proteome. As used herein an “isotope tag” refers to a chemical moiety having suitable chemical properties for incorporation of an isotope, allowing the generation of differentially tagged polypeptides in two samples. The isotope tag also has an appropriate composition to allow incorporation of a stable isotope at one or more atoms. A particularly useful stable isotope pair is hydrogen and deuterium, which can be readily distinguished using mass spectrometry, for example, 13C, 15N, 17O, 18O or 34S. Amino acid-specific methods such as ICAT™ have the advantage of reducing sample complexity but have the disadvantage of discriminating against proteins with low number of cysteines. Proteins can be also isotopically labeled at the C-termini of the tryptic peptides. One method of global labeling inserts an isotopic label via the molecule of water that is incorporated into peptides during cleavage of amide backbones by enzyme. While chymotrypsin and Asp-N incorporate only one 18O atom, trypsin, Glu-C or Lys-C can incorporate two 18O atoms into the C-termini of the resulting peptides. Moreover, 18O labels in the carboxylate groups of peptides and amino acids are resistant to back exchange. Thus under common conditions for liquid chromatography, electrospray ionization (ESI), and matrix-assisted laser desorption/ionization (MALDI), covalent bonds between oxygen atoms and carbonyl carbon in a C-terminal carboxylate group are stable. The practice of 18O-labeling is receiving increasing attention as a preferred method for heavy isotope labeling and several examples of its application have been published. It is common practice and considered advantageous to use the highest enrichment of H218O as possible in order to achieve the highest degree of labeling of each proteolytic fragment for a quantitative application of proteomics.

### BRIEF DESCRIPTION OF DRAWING

FIG. 1 is a flow chart for the identification of proteins by proteomic analysis using protein cleavage at aspartic acid (PCA) method.

FIGS. 2A, 2B, and 2C are MALDI-MS and MALDI-TOF/TOF analysis of BSA (Bovine Serum albumin) by PCA method. Spectra were obtained utilizing an Applied Biosystems 4700 Proteomics Analyzer™: Panel A; MALDI mass spectra (mass range 800-4300 Da); Panel B; Database search results by PMF with MASCOT™; Panel C; For MS/MS(MALDI-TOF/TOF) analyses of the 1723.86 Da monoisotopic peak, spectra were obtained by the accumulation of 5000 consecutive laser shots at a collision energy of 1 kV with air serving as the collision gas.

FIGS. 3A and 3B are MALDI-MS analysis of Ubiquitin by PCA method. Spectra were obtained utilizing Applied Biosystems 4700 Proteomics Analyser™: Panel A; MALDI mass spectra (mass range: 400-4300 Da); Panel B; Database search results by PMF (peptide mass fingerprint) with MASCOT™.

FIGS. 4A, 4B and 4C are database search results with MASCOT™: Protein sample; (BSA) Bovine Serum Albumin; Panel A; Database search result with MALDI-TOF mass list (above S/N 10) of BSA digested through PCA method. [Sequence coverage: 40%]; Panel B; Database search result with MALDI-TOF mass list (above S/N 10) of BSA digested by trypsin. [Sequence coverage: 32%]; Panel C; Database search result with MALDI-TOF mass list (above S/N 10) obtained by combination PCA method and tryptic digestion. PCA/trypsin method's database search result shows high sequence coverage [87%]

FIG. 5 shows detergent-free and Chaotropic reagent-free chemical digestion of membrane proteins. De-lipidated membrane protein aggregates obtained from mouse brain lipid raft were digested in PCA solution (15 (v/v) % of acetic acid (pH 2.0), 30 (v/v) % of acetonitrile, and TCEP (10 mM)) were incubated at 99° C. for 4 hrs by using PCR machine. The chemically digested membrane proteins were analyzed by SDS-PAGE, and visualized by CBB-staing.

FIG. 6 shows UV chromatogram from a reverse-phase HPLC separation of the hydrolysate obtained from PCA reaction of de-lipidated membrane protein aggregates. About 1 mg of the protein hydrolysate was injected. Pooled fractions were indicated by the circled number.

FIGS. 7A, 7B and 7C are MALDI-TOF/TOF detection of ubiquitin by PCA method: Panel A; MS spectra of ubiquitin processed by PCA method; Panel B; MS spectra of ubiquitin obtained by PCA-DMT (Differential Mass Tagging) method; Panel C and D; 1:1 and 1:2 mixture of PCA/PCA-DMT cleaved ubiquitin.

FIG. 8 shows the result of LC-MS/MS experiment using the hydrolysates obtained from sequential digestion of mouse brain lipid raft by PCA and Trypsin: Panel A; Total ion chromatogram of tryptic hydrolysate of each pooled fraction in FIG. 4. Each pooled fraction was indicated by circled number; Panel B; Extracted ion chromatogram at a marked time in panel A; Panel C; Tandem MS/MS result of the ion circled in panel B.

FIG. 9 is an assignment of the mass of peptide fragments of ubiquitin obtained by PCA method [sequence coverage is 100%]

FIGS. 10A and 10B are an assignment of the mass of observed peptide fragments of BSA (Bovine Serum Albumin) by PCA method. The peptide mass list obtained from MALDI-TOF and the identities of some peptide were verified from de novo sequencing by tandem MS.

FIGS. 11A and 11B are the selected list of mouse brain lipid raft proteins identified by LC-MS/MS of the hydrolysates obtained from sequential digestion by PCA and Trypsin.

### SUMMARY OF THE INVENTION

An objective of this invention is to provide the method, kit, and apparatus for acid hydrolysis of proteins, which can guarantee the strict specificity of cleavage at aspartyl residue without the production of unpredictable modification of the peptides.

The invention provides the optimal composition of reagents for acid hydrolysis of proteins, referred to herein as protein cleavage at aspartic acid (PCA), for protein identification and quantification. The identification of proteins in a given sample can be achieved by de novo sequencing of the peptides generated or by peptide mass fingerprinting from MS analysis results by accommodating newly developed rules of fragmentation.

The present invention includes the designing of apparatus for PCA, which is developed for incubating the solution above 95° C. with minimizing the loss of vapor pressure by heating the lid as well as bath simultaneously in the same temperature. The present method provides handy and simple procedure for processing of proteins prior to MS analysis comprising of just a few hours of incubation and sample dry. Furthermore, PCA can be used in combination with tryptic digestion to generate the peptides suitable for tandem MS analysis in order to get enough information for the detailed structural analysis of proteins.

The invention further provides methods for quantifying proteins in a sample by adopting the concept of ¹⁸O-labeling of proteins using H2¹⁸O during hydrolysis for comparative proteomics.

### DETAILED DESCRIPTION

The present invention provides a polypeptide hydrolyzing composition comprising an acid component, water miscible organic solvent and a reducing agent. The acid component is trifluoroacetic acid, phosphoric acid, propionic acid, HCl, o-iodobenzoic acid, glacial acetic acid, or any acid having buffering capacity near pH 2. Preferably, the acid component can be a mixture of trifluoroacetic acid, phosphoric acid, propionic acid, HCl, and o-iodobenzoic acid.

pH of hydrolyzing solution at time of reaction is in the range of 1.5 to 2.5 The hydrolyzing solution comprises at least 2 to 30 (v/v) % glacial acetic acid. The hydrolyzing solution comprises 15 (v/v) % glacial acetic acid, pH 2.0.

The water miscible organic solvent is Acetonitrile, DMF (Dimethyl formamide), DMSO (Dimethylsulfoxide), THF (Tetrahydrofurane), or an alcohol. The alcohol is methanol or ethanol. For example, the water miscible organic solvent is at least 5-70 (v/v) % Acetonitrile, preferably 30 (v/v) % Acetonitrile.

The reducing agent is TCEP (Tris(2-carboxyethyl)phosphine), DTT (Dithiothreitol), or beta-Mercaptoethanol. The reducing agent is phosphine compound which can work at acidic pH range (1.5-2.5) such as TCEP (Tris(2-carboxyethyl)phosphine). The reducing agent is at least 1 mM-1M TCEP (Tris(2-carboxyethyl)phosphine) or DTT (Dithiothreitol). The reducing agent is at least 10 mM TCEP (Tris(2-carboxyethyl)phosphine) or DTT (Dithiothreitol).

For example, the composition comprises about 2-30 (v/v) % acetic acid of the hydrolyzing solution, about 5-70 (v/v) % acetonitrile of the hydrolyzing solution, and about 1 mM-1M of TCEP ((Tris(2-carboxyethyl)phosphine). The composition comprises about 15 (v/v) % of acetic acid of the hydrolyzing solution, acetonitrile at an amount of about 5-70 (v/v) % of acetonitrile of the hydrolyzing solution, and about 1 mM-1M of TCEP ((Tris(2-carboxyethyl)phosphine). The composition comprises about 15 (v/v) % of acetic acid of the hydrolyzing solution, about 30 (v/v) % of acetonitrile of the hydrolyzing solution, and about 1 mM-1M of TCEP ((Tris(2-carboxyethyl)phosphine). The composition comprises about 15 (v/v) % of acetic acid of the hydrolyzing solution, about 30 (v/v) % acetonitrile of the hydrolyzing solution, and 10 mM of TCEP ((Tris(2-carboxyethyl)phosphine).

The composition may not include the water miscible organic solvent and a reducing agent. The composition may not include the water miscible organic solvent or the reducing agent.

The composition of the present invention can include a detergent which is OBG (octyl-beta-glucopyranoside) or SDS(Sodium dodecyl sulfate).

In addition, the present invention provides a method for hydrolyzing a polypeptide at an aspartic acid amino acid residue comprising contacting the polypeptide with a hydrolyzing solution of the present invention to obtain polypeptide fragments having aspartic acid residue at the N- or C-terminus and optionally determining amino acid sequence of resultant polypeptide fragments.

In an embodiment of the present invention, a method of determining amino acid sequence of a polypeptide comprises:

(i) hydrolyzing the polypeptide with the composition to obtain polypeptide fragments having aspartic acid residue at the N- or C-terminal ends of the fragments;

(ii) determining sequence of resultant polypeptide fragments; and

(iii) determining the sequence of the polypeptide by matching and connecting the sequences of the polypeptide fragments so as to obtain the full sequence of the polypeptide.

The sequence of polypeptide fragments is determined through mass spectrometry. Water is labeled with deuterium, or tritium, or ¹⁷O, or ¹⁸O labeled water. The hydrolysis of the polypeptide is carried out in a reaction temperature in the range of about 75 to 150° C. The container material for hydrolysis reaction is made of plastic which is made of polyethylene, polypropylene, high density polyethylene, or low density polyethylene. The reaction heat is created by micro wave, or ultra sonic wave.

The sequence of the polypeptide can be determined by a database search using a modified cleavage rule incorporating polypeptide fragments having aspartic acid residues at either the N- or C-terminal ends or both the N- and C-terminal ends. The database search is carried out with PCA database menu which has a cleavage rule and modification rule incorporating polypeptide fragments having aspartic acid residues at either the N- or C-terminal ends or both the N- and C-terminal ends.

In an embodiment of the present invention, a method of determining amino acid sequence of a polypeptide comprises:

(i) hydrolyzing the polypeptide with protease(s) to obtain polypeptide fragments;

(ii) hydrolyzing the composition obtained in step (i) with the composition to obtain polypeptide fragments having aspartic acid residue at the N- or C-terminal ends of the fragments;

(iii) determining sequence of resultant polypeptide fragments;

(iv) determining the sequence of the polypeptide by matching and connecting the sequences of the polypeptide fragments so as to obtain the full sequence of the polypeptide.

The sequence of the polypeptide can be determined by a database search using a modified cleavage rule incorporating polypeptide fragments having aspartic acid residues at either the N- or C-terminal ends or both the N- and C-terminal ends. The database search is carried out with PCA database menu which has a cleavage rule and modification rule incorporating polypeptide fragments having aspartic acid residues at either the N- or C-terminal ends or both the N- and C-terminal ends.

In an embodiment of the present invention, a kit for hydrolyzing polypeptide comprises (i) a container containing an acid solution and water; (ii) a container containing a water miscible organic solvent and a reducing agent. The reducing agent can be omitted.

The present invention is further explained in more detail with reference to the following examples. These examples, however, should not be interpreted as limiting the scope of the present invention in any manner.

1. Protein Cleavage at Aspartic Acid (PCA) Using Chemical Reagents

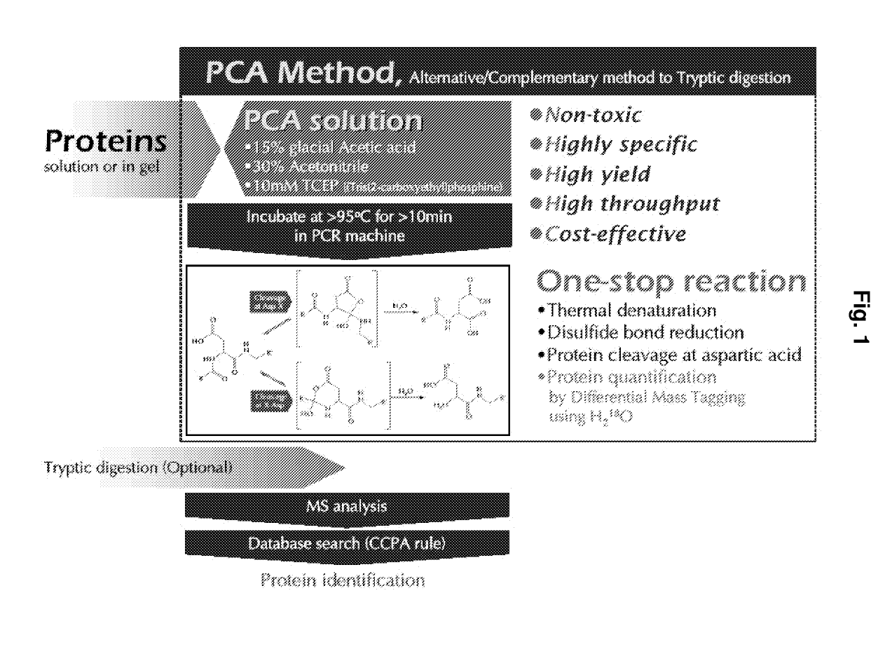

This invention (PCA) provides a superior method for the proteomic analysis of proteins than any other digestion method. As shown in FIG. 1, the proteins in-solution or in-gel can efficiently be cleaved to generate peptides using PCA solution, and the mass pattern and the sequence of amino acid of the resulting peptides can be analyzed by mass spectrometer. Proteins dissolved in solvents or in gel band are incubated at the temperature higher than 95° C. (Incubation at 99.9° C. is preferable) for more than 10 minutes in the presence of PCA solution.

1-1. The Composition of PCA Solution

1. Acid: trifluoroacetic acid, phosphoric acid, propionic acid, HCl, o-iodobenzoic acid, glacial acetic acid, Formic acid,

(Acid such as acetic acid that has a buffering capacity near pH 2 but do not bring about any unexpected modifications during reaction is preferable.)

2. Water miscible organic solvent: Acetonitrile, DMF (Dimethyl formamide), DMSO (Dimethylsulfoxide), THF (Tetrahydrofurane), any kinds of alcohol such as methanol and ethanol,

(The amount of acetonitrile is preferably 30 (v/v) %.)

3. Reducing agent for disulfide bond: TCEP (Tris(2-carboxyethyl)phosphine), DTT (Dithiothreitol),

(Reducing agent such TCEP that can work at acidic pH range is preferable.) In terms of yield, efficiency and specificity, reaction in the presence of 15% acetic acid (2.62 mM), 30 (v/v) % acetonitrile and TCEP (10 mM) is optimal for protein cleavage at aspartic acid and reduction of disulfide bond in proteins.

The sample was then cooled to room temperature and the reaction solution was dried in the same reaction tube without transferring to a new tube for speed-vac dry. The dried peptide extract was diluted with appropriate volume of 0.1 (v/v) % TFA. Desalting process for removing TCEP oxide (TCEPO) and other salts which can interfere with mass analysis can be done by the passage through μ-C18 ZipTips. The resulting peptides are analyzed by mass spectrometer.

1-2. Sample Test with BSA and Ubiquitin

1-2-1. BSA

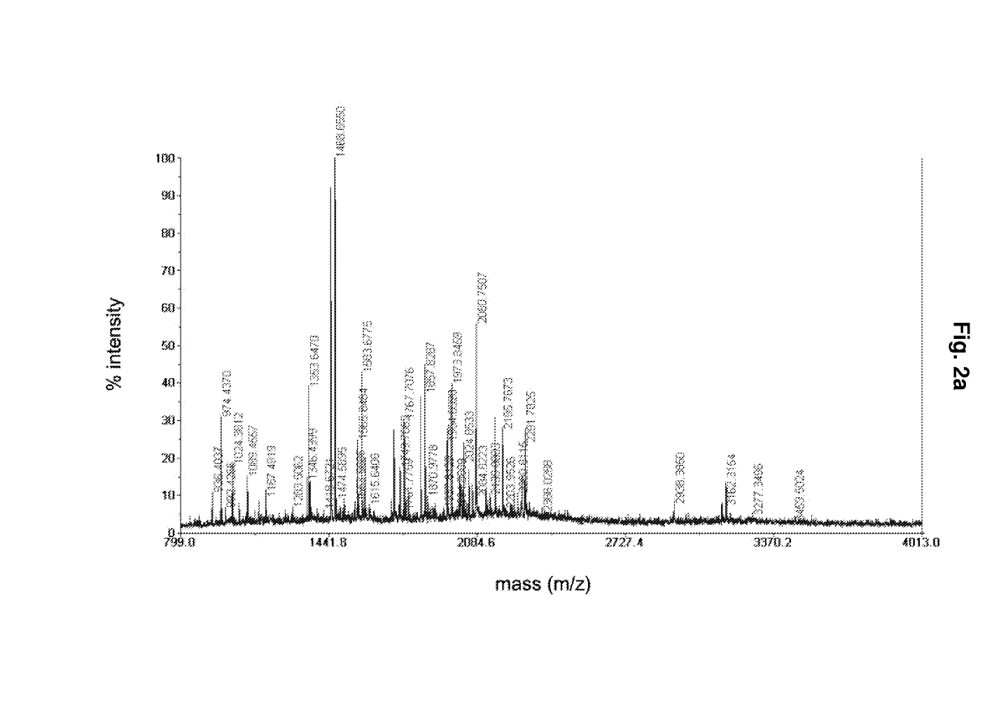

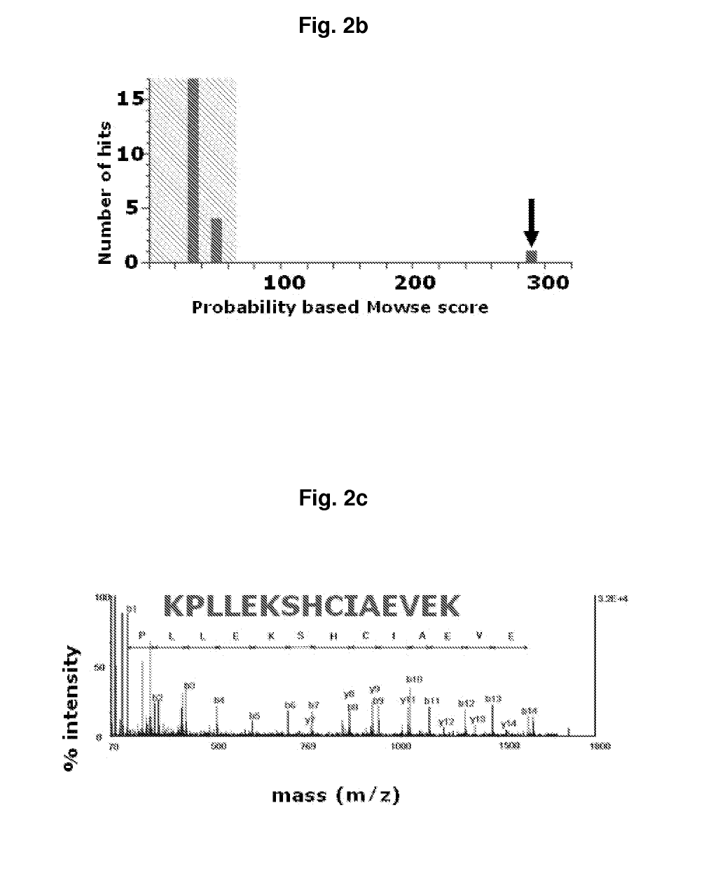

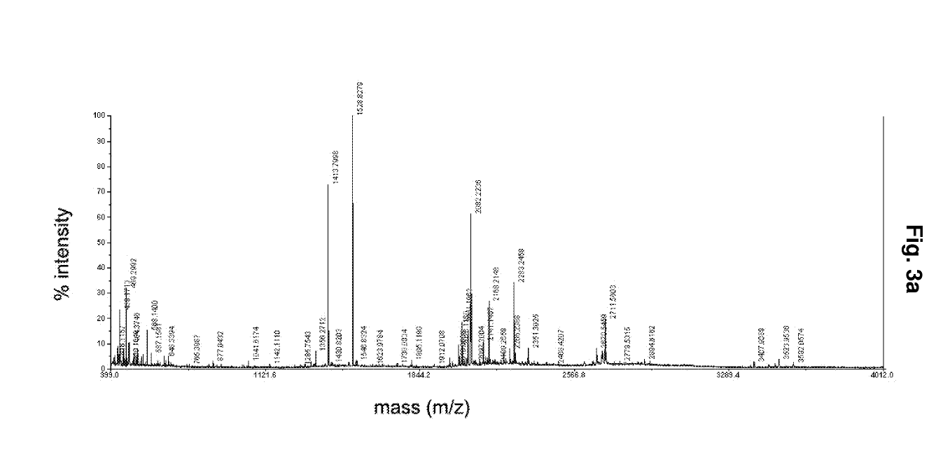

Bovine Serum Albumin (BSA, Calbiochem Catalog No. 126609) is used to verify the usefulness of PCA method for sample preparation. The reaction was carried out at 99.8° C. for 2 hrs in the presence of PCA solution (15 (v/v) % of acetic acid (pH 2.0), 30 (v/v) % of acetonitrile, and 10 mM TCEP). Any optional modifications except for pyro-glu E (N-term) and pyro-glu Q(N-term) were not observed in the mass spectra obtained. These optional modifications do not interfere with the identification of proteins by MASCOT™. During the course of reaction, 57 of peaks with satisfying signal-to-noise ratio are observed, which can be assigned to expected products generated by the cleavage of BSA at aspartyl residues. Of the BSA sequence, 43% was recovered directly by the accommodation of PCA method (FIG. 2). More cysteine-containing peptides are retrieved with the inclusion of TCEP (Tris(2-carboxyethyl)phosphine) as a reducing agent instead of DTT (Dithiothreitol) in the PCA solution. Lots of DTT (dithiothreitol) are needed for disulfide bonds cleavage between peptides in the acidic hydrolysis condition, because the optimal pH for reducing reaction of DTT (dithiothreitol) is slightly basic. TCEP (Tris(2-carboxyethyl)phosphine) is a choice of reducing agent which can work at the range of pH optimal for acid hydrolysis of proteins.

1-2-2. Ubiquitin

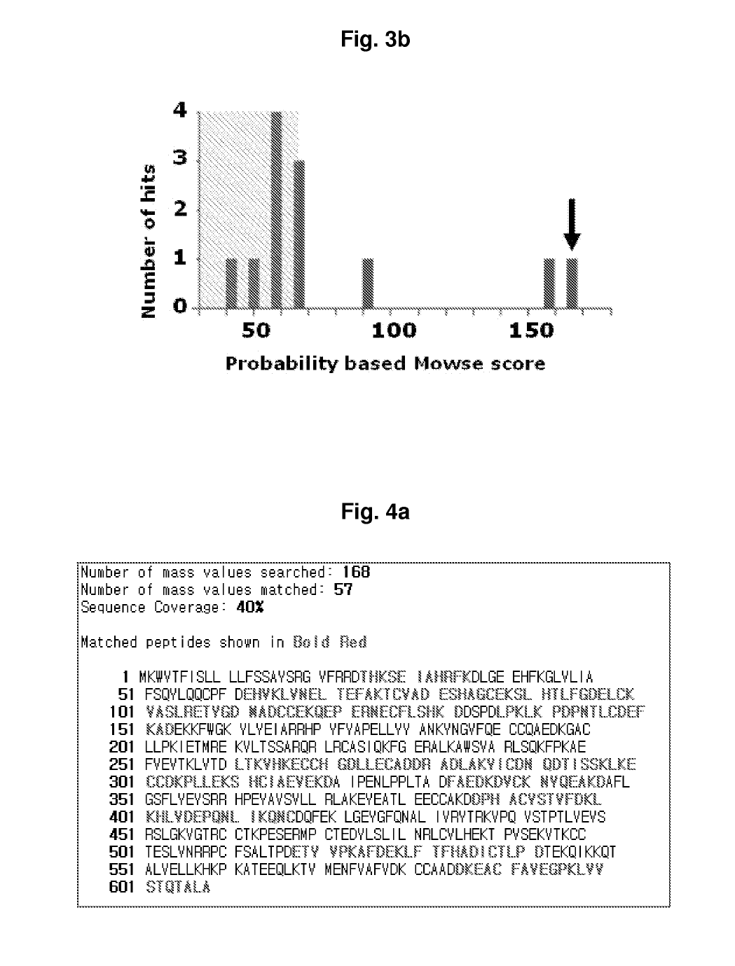

We used ubiquitin (Sigma Catalog No. U6253) to test the feasibility of the PCA method for small size proteins. The reaction was carried out at 99.9° C. for 2 hrs in the presence 15 (v/v) % of acetic acid having pH 2.0 and 30 (v/v) % of acetonitrile. For ubiquitin, TCEP is not included in the reaction mixture due to the lack of disulfide bond in ubiquitin. Cleavage of ubiquitin with the PCA method resulted in perfect coverage, 100% (FIG. 3). Tryptic digestion retrieved about 82% of sequence of ubiquitin, but another variant of ubiquitin was picked up by MASCOT™.

2. PCA in Combination With Enzymatic Digestion

We have explored the usefulness of PCA method in combination with enzymatic digestion. Trypsin is a common choice of enzyme widely used in the digestion of proteins. Cleavage at aspartyl residue (D) can be useful in dealing with the samples for which enzymes have poor accessibility for proteolytic attack and amino acids for cleavage are scarce or lacking.

2-1. Sample Test With BSA for Multiple Digestion

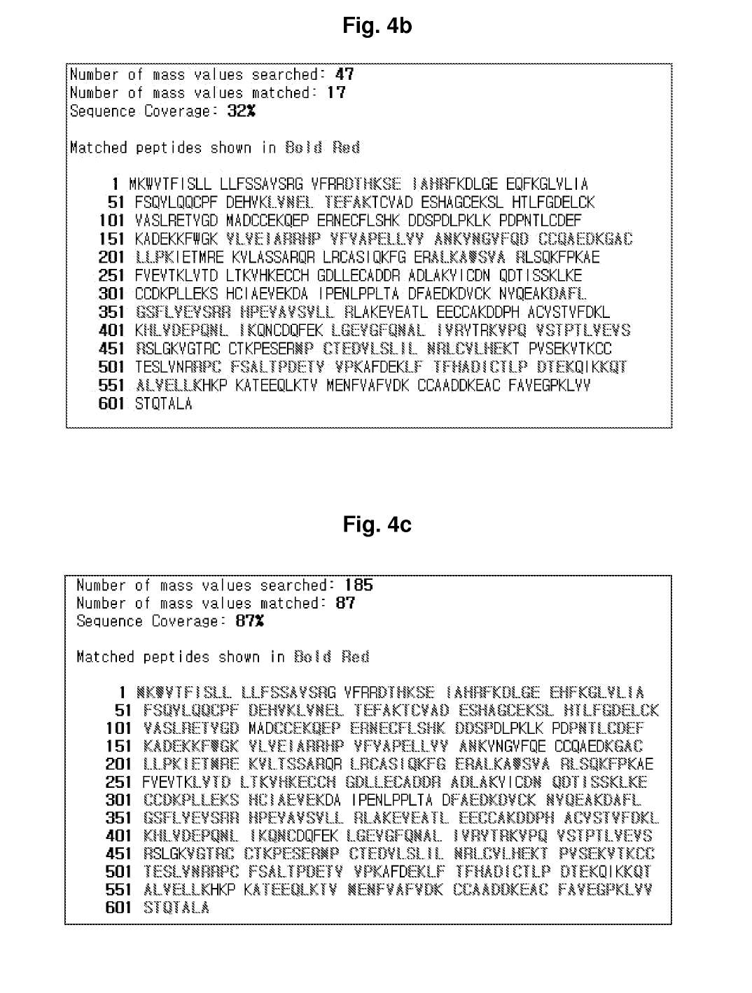

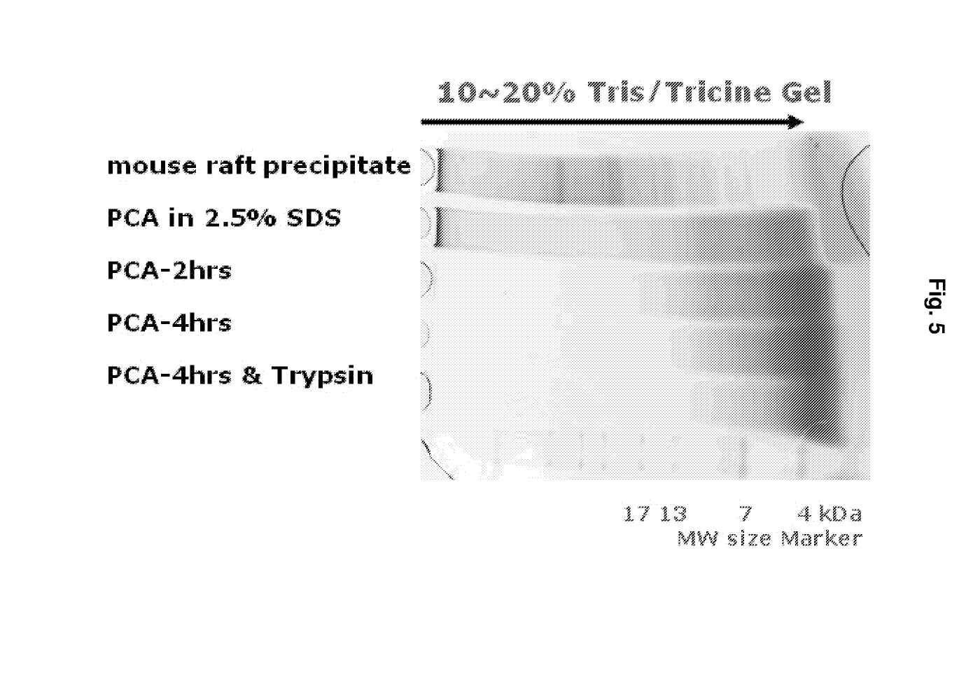

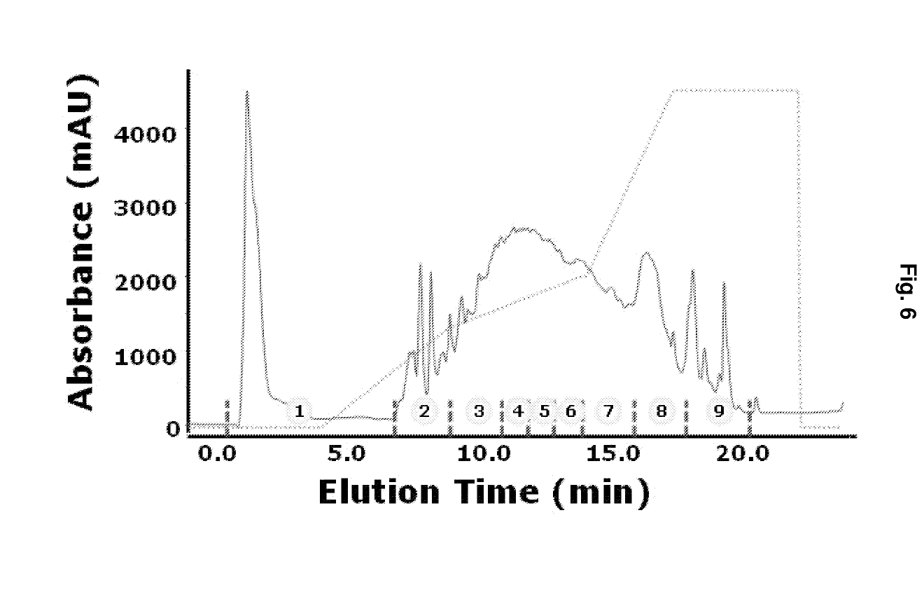

BSA (bovine serum albumin, 10 pmole) was dissolved in 50 mM ammoniumbicarbonate solution to make a concentration of 1-5 μM. Aliquots from protein solutions were thermally denatured by incubating at 90° C. for 20 min. Following incubation, the proteins were transferred to an ice-water bath to quench the denaturation process. Thermally denatured protein samples were enzymatically digested with sequencing-grade modified trypsin at 37° C. for 12 hrs. Digested sample in 50 mM ammoniumbicarbonate was transferred into reaction tube and then heated at 99.9° C. for 30 min to remove ammoniumbicarbonate by evaporation. Following heating, the dried samples dissolved in PCA solution (15 (v/v) % of acetic acid (pH 2.0), 30 (v/v) % of acetonitrile, and TCEP (10 mM)) were heated at 99.9° C. for 2 hrs. The sample was then cooled to room temperature and the reaction solution was dried at 99.9° C. in the same reaction tube. The dried peptide extract was diluted with 5 μL 0.1 (v/v) % TFA and desalted with μ-C18 ZipTips. The peptides bound to the ZipTip were eluted out sequentially in 5 μL each of 20 (v/v) %, 50 (v/v) % and 80 (v/v) % ACN in 0.1 (v/v) % HAc (acetic acid) solution or just 50 (v/v) % ACN/0.1 (v/v) % HAc. The sequentially peptide solution was dried to reduce its volume to about 5 μL and a matrix solution was added for MALDI-TOF analysis. The purpose of using ZipTip is to remove salts from the sample prior to MS detection. This approach was applied to characterization of proteins such as BSA (bovine serum albumin) resulting in improved identification with high sequence coverage (87%) comparing with 40% and 32% obtained by PCA method and tryptic digestion, respectively as shown in FIG. 4.

2-2. Sequential Digestion of Membrane Proteins With Chemical and Enzymatic Method

Proteomic analysis of membrane proteins has been indispensable and challenging subject for understanding of diverse signaling networks and for discovering targets for a given disease. Most of researchers have relied on classical proteomic method consisted of two-dimensional electrophoresis for separating proteins prior to enzymatic digestion and mass spectrometric analysis, while only few studies have been reported by accommodation of gel-free LC-MS/MS technologies. However, even gel-free LC-MS/MS technologies depend on detergents or chaotropic reagents to isolate the integral proteins from membrane fraction. Hereby, we present novel gel-free and detergent-free shotgun proteomic method for analyzing membrane proteins. By the application of our proprietary technology (CCPA method, Chemical Cleavage of Proteins at Aspartic acid), de-lipidated aggregate of mouse lipid raft proteins were successfully digested into peptides.

2-2-1. Lipid Raft Preparation From Mouse Brain

Lipid raft was selected for proving the usefulness of chemical digestion, because lots of proteomic studies have been performed to elucidate the composition of proteins in lipid raft. Lipid raft was prepared as described in Proteomics 2004, 4, 3536-3548. Mouse brains were homogenized 20 times with a tight Dounce homogenizer (Kontes, Vineland, N.J., USA) in the lysis buffer (1 (v/v) % Triton X-100, 25 mM HEPES, pH 6.5, 150 mM NaCl, 1 mM EDTA, 1 mM PMSF, and protease cocktail (Roche Molecular Biochemicals, Indianapolis, Ind., USA)), and incubated at 40 C. for 20 min. The extract was mixed with 2.5 M sucrose, transferred to an SW41 centrifuge tube, and overlaid with 30 (w/v) % sucrose solution and 5 (w/v) % sucrose solution containing 25 mM HEPES, pH 6.5, and 150 mM NaCl. The discontinuous sucrose gradients were centrifuged for 18 h at 40 C. in an SW41 rotor at 39 000 rpm. The gradient was fractionated into 12 fractions from the bottom to the top. The lipid raft fractions were washed with washing buffer (25 mM HEPES, pH 7.4, 150 mM NaCl) by ultracentrifugation (20 000 rpm, 30 min, 47 C.), and suspended with 50 mM sodium bicarbonate. By the treatment with acetone and successive washing steps, the precipitate of membrane was obtained. The resulting protein precipitated was dried.

2-2-2. PCA Reaction of De-Lipidated Membrane Proteins

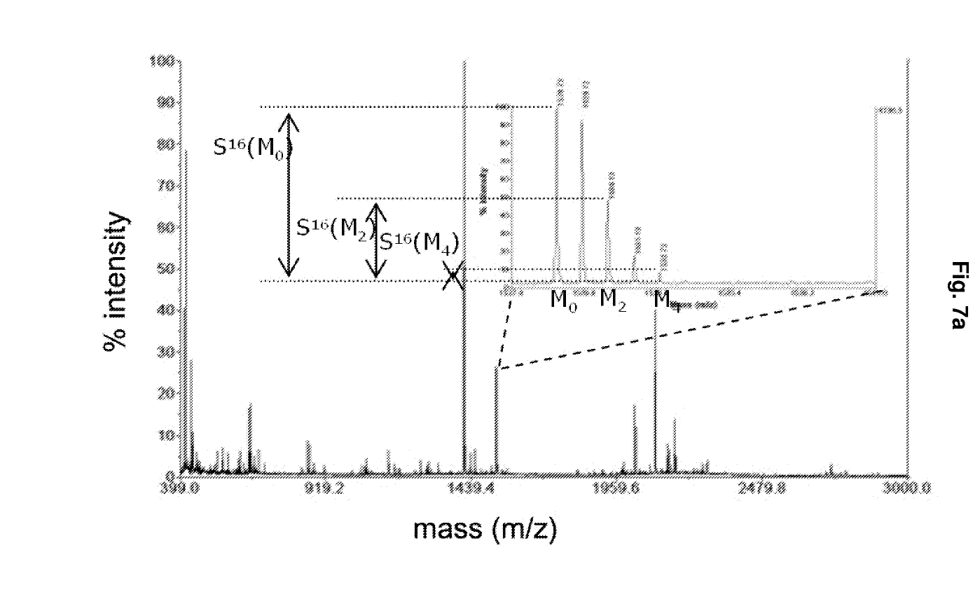

The dried material dissolved in PCA solution (15 (v/v) % acetic acid (pH 2.0), 30 (v/v) % acetonitrile, and TCEP (10 mM)) were incubated at 99° C. for 4 hrs by using PCR machine. The digestion of membrane proteins into peptides were confirmed by the visualization of proteins or peptides following Tris-Tricine gel electrophoresis as shown in FIG. 5.

2-2-3. Separation of PCA-Digested Membrane Proteins

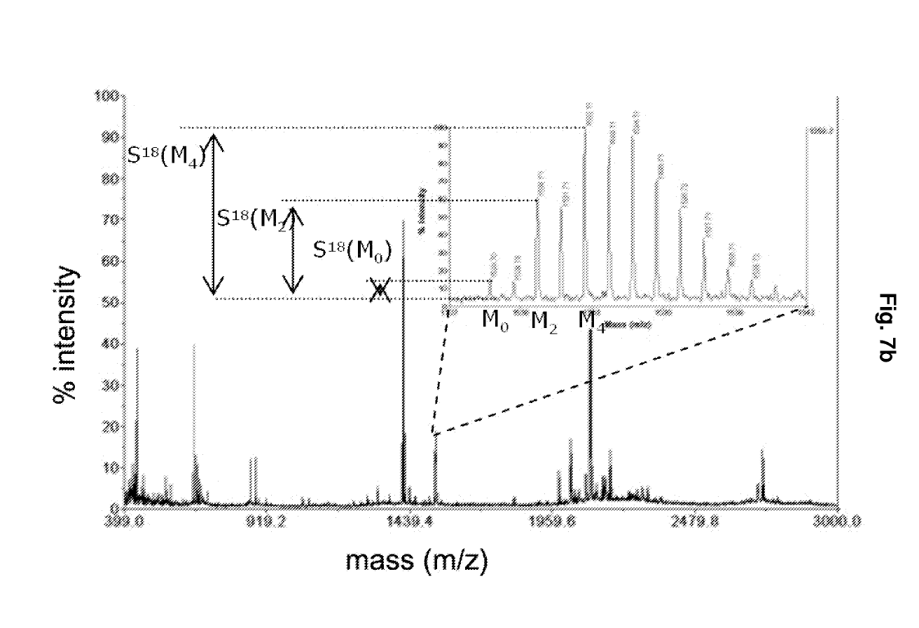

Chemically digested membrane proteins were separated by using reverse-phase column chromatography (Chromolith-C18, Merck) as shown in FIG. 6. The fractions from column were pooled to bring about 9 fractions. Nine fractions were dried separately.

2-2-4. Tryptic Digestion of PCA-Digested Membrane Proteins

The dried 9 fractions were incubated with 1/50 amount of Trypsin in the presence of 50 mM Ammonium Bicarbonate at 37° C. for 12 hrs. The resulting peptides were analyzed by LC-MS/MS.

3. PCA-DMT (Differential Mass Tagging)

For the quantitative analysis of the samples, two samples are labeled with a chemically identical but isotopically different tagging by using PCA solution containing 16O-water or 18O-water. During the course of reaction, the exchange of one or two atoms of oxygen can take place, thereby bring about the difference of molecular mass of 2 or 4 Da which can be discriminated in MS analysis. In PCA-DMT method, relative quantification for protein can be achieved by comparing the signal intensities of monoisotopic mass obtained by the chemical digestion of protein in the presence of 116O-water with those obtained by the inclusion of 18O-water in PCA solution.

3-1. Sample Test with Ubiquitin

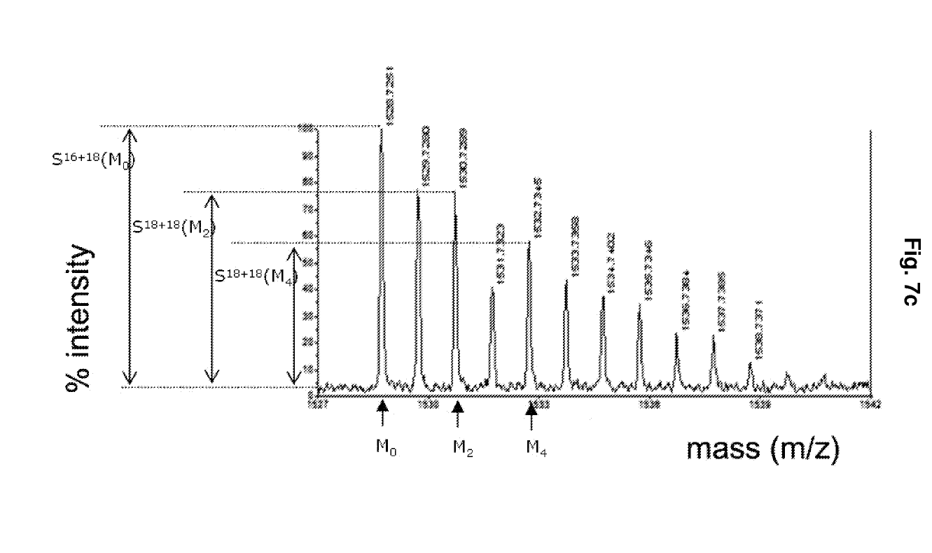

Proteins dissolved in water or dried gel band are heated at 99.9° C. for 2 hrs in the presence of PCA solution (15 (v/v) % acetic acid (pH 2.0), 30 (v/v) % acetonitrile, and 10 mM TCEP). For differential mass tagging, 16O-water was replaced by 18O-water. The sample was then cooled to room temperature and the reaction solution was dried at 99.9° C. in reaction tube and dissolved with 5 μL 0.1 (v/v) % TFA, and salt was removed by desalting process using μ-C18 ZipTips. The peptides bound to the ZipTip were eluted out sequentially in 5 μL each of 20 (v/v) %, 50 (v/v) % and 80 (v/v) % ACN in 0.1 (v/v) HAc(acetic acid) solution or just 50 (v/v) % ACN/0.1 (v/v) % HAc. The sequentially peptide solution was dried to reduce its volume to about 5 μL and a matrix solution was added for MALDI-TOF analysis. One of the examples is shown in FIG. 7. Accurate mass measurement of the monoisotopic peak (MH+ (obs) 1528.83, MH+ (calc) 1528.80) allowed this peptide to be assigned as QQRLIFAGKQLED (Pyro-glu(N-term)) and the shift of molecular mass by 2 or 4 was also observed indicating incorporation of one or two 18O atoms in the carboxylate group at the C-termini. Mass spectra was obtained in a mass accuracy of 10 ppm using the MALDI-TOF (ABI 4700 analyzer™).

When m/z value (value of x-axis in mass spectra) of monoisotopic mass is described as Mo, the series of isotopic mass is herein presented as M1, M2, M3, M4 in ascending order according to the difference in molecular mass. The signal intensity (value of y-axis in mass spectra) of a given m/z is described as S16(M0), S16(M1), S16(M2), S16(M3), and S16(M₄). Number 16 in this description indicates the atomic mass of oxygen atom of water molecule used in the reaction of chemical cleavage. According to this, signal intensity of monoisotopic mass obtained through the chemical cleavage in the case of using 18O-water is described as S18(M0), and that obtained by mixing the products prepared from separate chemical cleavage reaction in the presence of 16O-water or 18O-water is presented as S16+18(M0). K1 indicates the coefficient for the incorporation of one atom of oxygen in chemical cleavage reaction done in the presence of 18O-water, and K2 for the incorporation of two atoms of oxygen. By calculating the sum of K1 and K2, relative comparison of the amounts of proteins in two different samples can be achieved. In mathematical terms, K1 and K2 can be extracted from the formula as followed.

S16+18(Mx)=S16(Mx)+K1*S16(Mx−2)+K2*S16(Mx−4)

[m/z value should not be less than 0]

S16+18(M2)=S16(M2)+K1*S16(M0)+K2*S16(M−2)=S16(M2)+K1*S16(M0)

K1=(S16+18(M2)−S16(M2))/S16(M0)

S16+18(M4)=S16(M4)+K1*S16(M2)+K2*S16(M0)

K2=(S16+18(M4)−S16(M4)−K1*S16(M2))/S16(M0)

From the mass spectra in FIG. 7, mixing ratio of the peptides obtained by the chemical cleavage reaction of ubiquitin in the presence of 16O-water and 18O-water can be calculated according to the formula described above.

K1=(5963.09−0.49389*8251.5.6)/82515.6=0.2287

K2=(4966.49−(0.06472*8251.56+0.49389*1887.73))/8251.6=0.4242

K1+K2=0.6529

The ratio of the peptides obtained by the chemical cleavage reaction of ubiquitin in the presence of 16O-water and 18O-water is calculated as 1:0.65. The difference of calculated ratio from theoretical one can be due to the usage of 18O-water containing around 5% of 16O-water, the possible contamination of 16O-water in other reagents such TCEP and sample (Ubiquitin), and incorporation of 16O-water from air during the course of reaction.

4. Sample Preparation for MALDI MS Analysis.

The dried peptide extract was suspended in 5 μL 0.1 (v/v) % TFA and desalted with μ-C18 ZipTips. The peptides bound to the ZipTip were eluted out sequentially in 5 μL each of 20 (v/v) %, 50 (v/v) % and 80 (v/v) % acetonitrile in 0.1 (v/v) % acetic acid solution or just 50 (v/v) % acetonitrile/0.1 (v/v) % acetic acid. The sequentially peptide solution was dried to reduce its volume to about 5 μL and a matrix solution was added. The purpose of using ZipTip is to remove salts form the samples prior to MS detection, but it does not completely remove the n-OG, a nonionic detergent. The two-layer sample deposition method with A-CHCA as matrix was used in the MALDI-MS analysis. The first layer was prepared as a 20 mg/mL α-CHCA solution in 20 (v/v) % methanol/acetone α-CHCA and the second layer with a saturating solution of matrix in 30 (v/v) % methanol/water. The second layer was added to the Zip-Tipped peptide mixture for producing the ratio of matrix to analyte to 4:1 and the mixture vortexed. After 0.5 μL of the first layer was deposited on the sample probe and air-dried, 0.5 μL of the second layer was deposited on top of the first layer, allowed to air-dry and washed twice with 1 ρL water.

5. MALDI-Mass Spectrometry

A MALDI-TOF mass spectrometer with 4700 Proteomics Analyzer™ (Applied Biosystems) was used to acquire the mass spectra. The matrix solution was a 10 mg of α-CHCA in 50 (v/v) % acetonitrile in water. Aliquots of 0.5 μL of the peptide mixture and 0.5 μL of the matrix solution were mixed on the sample plate and air-dried prior to analysis.

6. MALDI Tandem Mass Spectrometry (MS/MS)

All MS/MS data from the TOF/TOF (4700 Proteomics Analyzer™) was acquired using the default 1 kv MS/MS method following manufacturer's instruction. MS/MS data acquisition form the plates (LC-MALDI plates) on which the LC eluent had been spotted by the Probot™ was performed in a four step process. First, MS spectra were recorded from each of the six calibration spots, and the default calibration parameters of the instrument and the appropriate model of plate model were updated. Second, MS spectra were recorded for all 144 sample spots on that plate. Each spectrum was generated by accumulating the data from 750 laser shots using the newly updated default calibration settings. Third, the 144 MS spectra were analyzed using the Peak Picker software supplied with the instrument. Spectral peaks that met the threshold criteria and were not on the exclusion list were included in the acquisition list for the MS/MS portion of the experiment. The threshold criteria were set as follows: mass range: 650 to 4000 Da; minimum cluster area: 500; minimum signal-to-noise (S/N): 10; Peaks/spot: 30; maximum precursor gap: 200 ppm; maximum fraction gap: 4. A mass filter excluding matrix cluster ions was applied. An XML file was generated that contains the list of the precursor masses selected for MS/MS and their corresponding spot numbers. Lastly, the list was imported into the 4700 Explorer software batch editor, and MS/MS spectra were recorded using air as the collision gas with 1 kV collision energy setting. During MS/MS data acquisition, a method with a stop condition was used. In this method, a minimum of 750 shots (6 sub-spectra accumulated from 125 laser shots each) and a maximum of 2000 shots were allowed for each spectrum. The accumulation of additional laser shots was halted whenever at least 10 ions with a S/N of at least 10 were present on the accumulated MS/MS spectrum, in the region from m/z 400 to 90% of the precursor mass.

7. LC-MS/MS Analysis of Double-Digested Lipid Raft Proteome

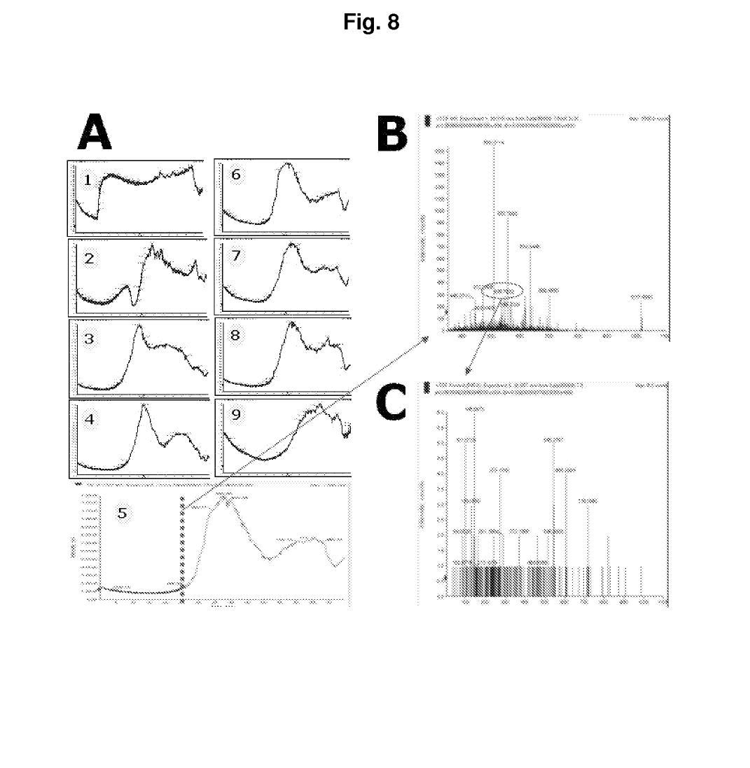

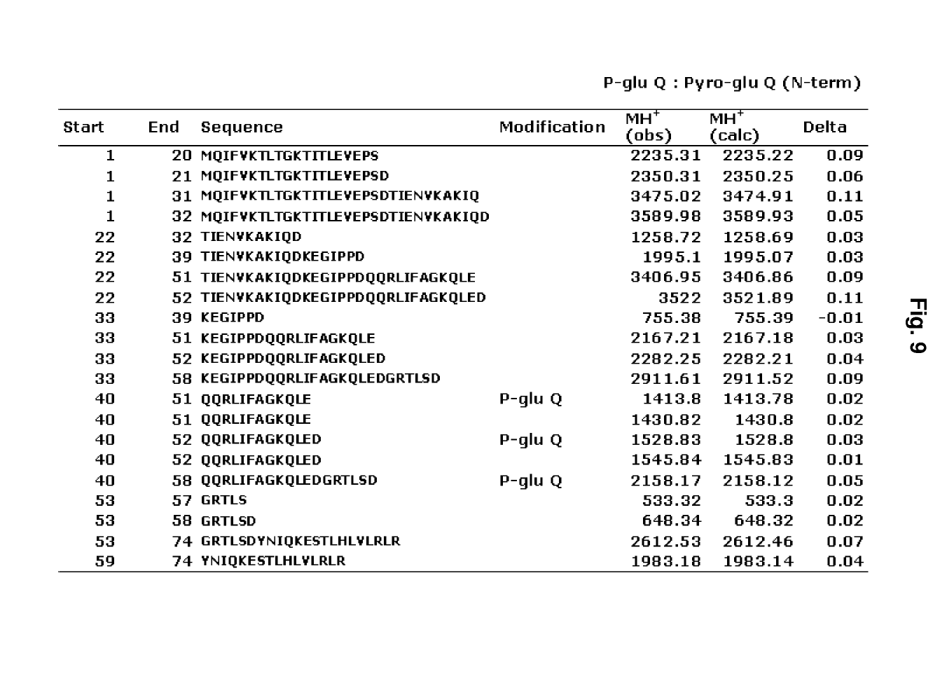

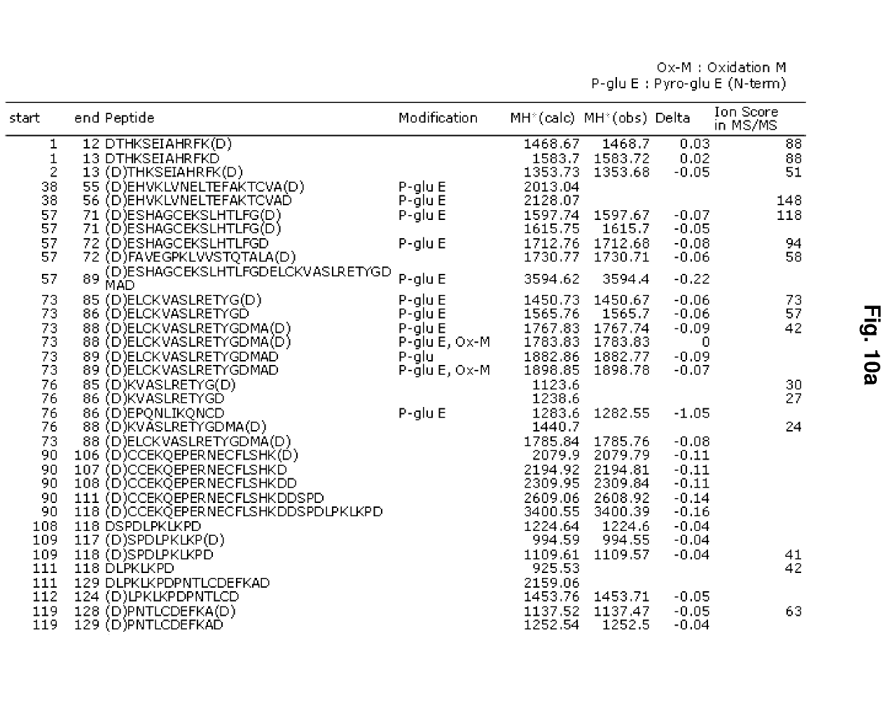

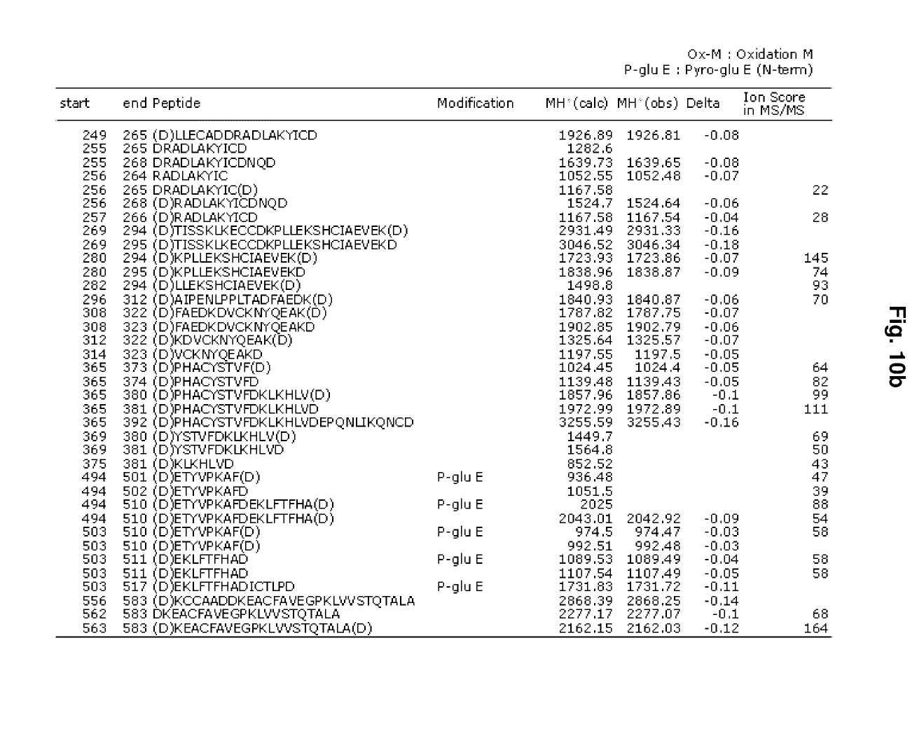

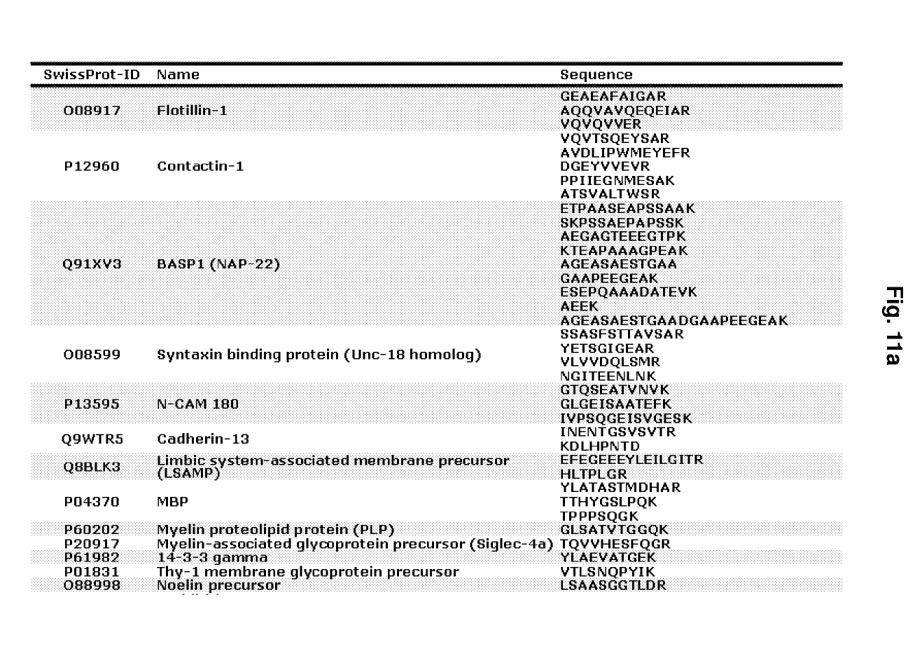

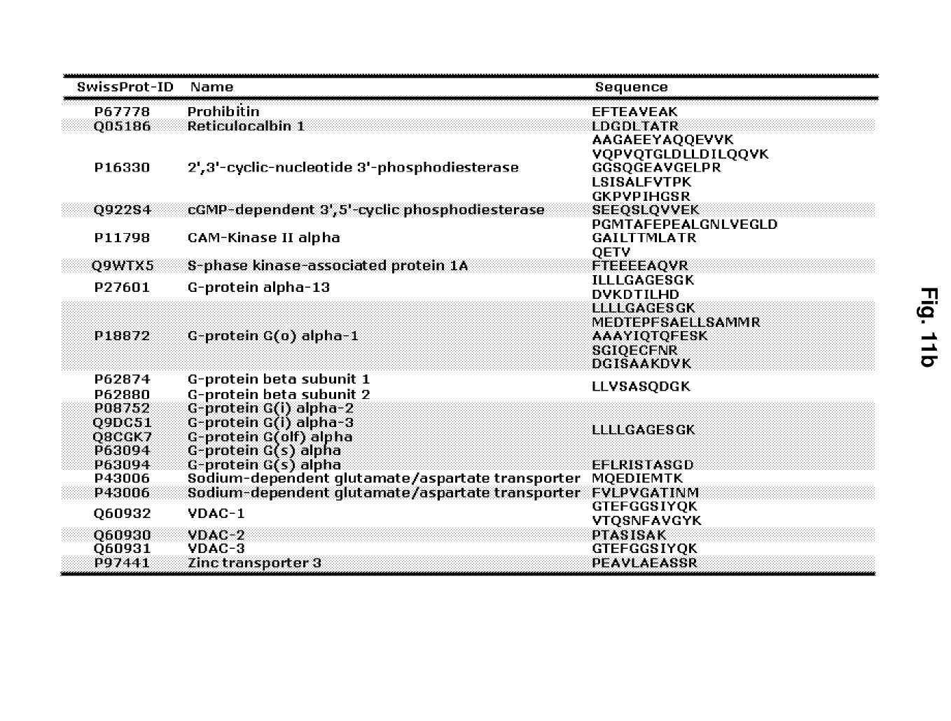

PCA/Trypsin-cleaved peptides were eluted from the nano LC system, Agilent 1100 (Agilent), and ions were sprayed directly into the orifice of a QSTAR-XL quadrupole time-of-flight (TOF) hybrid MS (PE-Sciex, Thornhill, Ontario, Canada). The results of LC-MS/MS experiments for the hydrolysate obtained from sequential digestion of mouse brain lipid raft by PCA and Trypsin were as shown in FIG. 8. Proteins were identified by LC/MS/MS by information-dependent acquisition of fragmentation spectra for multiply charged peptides that were then searched against the Human International Protein Index database (ftp://ftp.ebi.ac.uk/pub/databases/IPI/current/MOUSE) by using MASCOT (Matrix Science, London). The following search parameters were used in all MASCOT searches: maximum of two missed CCPA-trypsin cleavage, cysteine carbamidomethylation, methionine oxidation, and a maximum 0.2-Da error tolerance in the MS and 0.1-Da in the MS/MS data. Significant matches with the highest MOWSE scores were considered potential identification. All other hits were manually verified by using accepted rules for peptide fragmentation in a quadrupole-TOF hybrid MS. The selected subset of mouse brain lipid raft proteins was listed in FIG. 11. LC-MS/MS analysis of resulting peptides proteins brought about the identification of lots of bona fide membrane proteins such as G-proteins, adhesion molecules, channels/transporter, signaling proteins (CAM-Kinase and phosphodiesterase), and flotillin (a kind of raft marker protein).

8. Mass Spectra Interpretation and Database Searching.

The rule by which peptides are generated from protein was modified following instructions in the user's manual of MASCOT™ so that PCA cleavage rule at both Asp-X and X-Asp was reflected in the MASCOT™ program. The modifications of peptides which is observed in tryptic digestion, such as methionine oxidation, Pyro-glu E(N-term), Pyro-glu Q(N-term) were considered for database searching. The algorithm developed in this invention can be realized for any other programs developed for the analysis of mass spectra.

9. Kit for PCA Proteomics

Kit for PCA method can be supplied as a set of solution (solution A and solution B) and container designed for optimal heat transfer and for minimizing sample loss during chemical reaction (thin-wall tube for PCR reaction) is highly recommended. The reaction should be carried out in the heating apparatus designed for minimizing the loss of vapor pressure by heating the lid as well as bath simultaneously in the same temperature such as PCR machine for better performance.

Total reaction mixture (150 μL); Solution A (45 μL)+solution B (150 μL)

A. Solution A; 10 mM TCEP (Tris(2-carboxyethyl)phosphine) in Acetonitrile

B. Solution B; 21.4 (v/v) % glacial acetic acid (acid) in Water

Mixture of peptide can be included as internal mass standard. For differential mass tagging, 21.4 (v/v) % acetic acid in 18O-Water can be used.

The method described in this invention can be modified in numerous ways by a specialist with a full understanding of the fundamental principles. Therefore, the invention is not restricted to the aforementioned examples. Other types of mass instrument can be used for identification and quantification of proteins and peptide with specific cleavage rule and modification rule on the basis of database search algorithm.

The cited references are as follows:

1. Aiqun Li et al. Anal Chem. 2001, 73, 5395-5402, Chemical Cleavage at Aspartyl Residues for Protein Identification

2. Peter Roepstorff et al. Anal Chem. 1999, 71, 919-927, Use of Vapor-Phase Acid Hydrolysis for Mass Spectrometric Peptide Mapping and Protein Identification

3. Zee-Yong Park et al. Anal Chem. 2000, 72, 2667-2670, Thermal Denaturation: A useful Technique in Peptide Mass Mapping

4. Steven L. Cohen et al. Anal. Chem. 1996, 68, 31-37, Influence of Matrix Solution Conditions on the MALDI-MS Analysis of Peptides and Proteins

5. Bart A. van Montfort et al. J. Mass Spectrom. 2002, 37, 322-330, Improved in-gel approaches to generate peptide maps of integral membrane proteins with matrix-assisted laser desorption/ionization time-of fanlight mass spectrometry

6. J. Otte et al, J. Agric. Food Chem. 2000, 48, 2443-2447, Identification of Peptides in Aggregates Formed during Hydrolysis of b-Lactoglobulin B with a Glu and Asp Specific Microbial Protease

7. Adrianne Kishiyama et al, Anal. Chem. 2000, 5431-5436, Cleavage and identification of Proteins: A Modified Aspartyl-Prolyl Cleavage

8. Cornelia Koy et al. Proteomics 2003, 3, 851-858, Matrix-assisted laser desorption/ionization-quadrupole ion trap-time of flight mass spectrometry sequencing resolves structures of unidentified peptides obtained by in-gel tryptic digestion of haptoglobin derivatives from human plasma proteomes.

9. Melanie Lin et al. Rapid Commun. Mass Spectrum. 2003, 17, 1809-1814, Intact protein analysis by matrix-assisted laser desorption/ionization tandem time-of-flight mass spectrometry.

10. Xudong Yao et al. Anal. Chem. 2001, 73, 2836-2842, Proteolytic 18O Labeling for Comparative Proteomics: Model Studies with Two Serotypes of Adenovirus.

11. Marcus Bantscheff et al, Rapid Commun. Mass Spectrum. 2004, 18, 869-876, Femtomol sensitivity post-digest 18O labeling for relative quantification of differential protein complex composition.

12. Kenneth L. Johnson et al. J Am Soc Mass Spectrom 2004, 15, 437-445, A Method for Calculating 16O/18O Peptide Ion Ratios for the Relative Quantification of Proteomes.

17. Y. Karen Wang et al, Anal. Chem. 2001, 73, 3742-3750, Inverse 18O Labeling Mass Spectrometry for the Rapid Identification of Marker/Target Proteins.

18. Schnolze, M.; Jedrzejewski, P.; Lehmann, W. D. Electrophoresis 1996, 17, 945-953,

19. Methods in ENZYMOLOGY Vol 91, Enzyme Structure Part 1, 324-332, Cleavage at Aspartic Acid

20. Methods in ENZYMOLOGY Vol 4, Enzyme Structure, 255-263, Cleavage at Aspartic Acid

21. Gargi Choudhary et al. Journal of Proteome Research 2003, 2, 59-67

1. A polypeptide hydrolyzing composition comprising water and at least an acid component selected from the group consisting of trifluoroacetic acid, phosphoric acid, propionic acid, HCl, o-iodobenzoic acid, glacial acetic acid, and an acid having buffering capacity near pH 2.

2. The composition according to claim 1, wherein the composition further comprises at least one selected from the group consisting of a water miscible organic solvent and a reducing agent.

3. The composition according to claim 1, wherein the acid component is a mixture of trifluoroacetic acid, phosphoric acid, propionic acid, HCl, and o-iodobenzoic acid.

4. The composition according to claim 1, wherein the pH of the hydrolyzing composition at time of reaction is in the range of 1.5 to 2.5

5. The composition according to claim 1, wherein the hydrolyzing composition comprises at least 2 to 30 (v/v) % of glacial acetic acid.

6. The composition according to claim 1, wherein the hydrolyzing composition comprises 15 (v/v) % of glacial acetic acid having pH 2.0.

7. The composition according to claim 2, wherein the water miscible organic solvent is Acetonitrile, DMF (Dimethyl formamide), DMSO (Dimethylsulfoxide), THF (Tetrahydrofurane), or an alcohol.

8. The composition according to claim 7, wherein the alcohol is methanol or ethanol.

9. The composition according to claim 2, wherein the water miscible organic solvent is Acetonitrile.

10. The composition according to claim 2, wherein the water miscible organic solvent is at least 5-70 (v/v) % of Acetonitrile.

11. The composition according to claim 2, wherein the water miscible organic solvent is 30 (v/v) % of Acetonitrile.

12. The composition according to claim 2, wherein the reducing agent is TCEP (Tris(2-carboxyethyl)phosphine), DTT (Dithiothreitol), or beta-Mercaptoethanol.

13. The composition according to claim 2, wherein the reducing agent is a phosphine compound which can work at an acidic pH range (1.5-2.5) such as TCEP (Tris(2-carboxyethyl)phosphine).

14. The composition according to claim 2, wherein the reducing agent is at least 1 mM-1M TCEP (Tris(2-carboxyethyl)phosphine) or DTT (Dithiothreitol).

15. The composition according to claim 2, wherein the reducing agent is at least 10 mM TCEP (Tris(2-carboxyethyl)phosphine) or DTT (Dithiothreitol).

16. The composition according to claim 2, wherein the composition comprises acetic acid at an amount of about 2-30 (v/v) % of the hydrolyzing composition, acetonitrile at an amount of about 5-70 (v/v) % of the hydrolyzing composition, and about 1 mM-1M of TCEP ((Tris(2-carboxyethyl)phosphine).

17. The composition according to claim 2, wherein the composition comprises acetic acid at an amount of about 15 (v/v) % of the hydrolyzing composition, acetonitrile at an amount of about 5-70 (v/v) % of the hydrolyzing composition, and about 1 mM-1M of TCEP ((Tris(2-carboxyethyl)phosphine).

18. The composition according to claim 2, wherein the composition comprises acetic acid at an amount of about 15 (v/v) % of the hydrolyzing composition, acetonitrile at an amount of about 30 (v/v) % of the hydrolyzing composition, and about 10 mM-1M of TCEP ((Tris(2-carboxyethyl)phosphine).

19. The composition according to claim 2, wherein the composition comprises acetic acid at an amount of about 15 (v/v) % of the hydrolyzing composition, acetonitrile at an amount of about 30 (v/v) % of the hydrolyzing composition, and 10 mM of TCEP ((Tris(2-carboxyethyl)phosphine).

20. The composition according to claims 1, wherein the composition further comprises a detergent.

21. The composition according to claim 20, wherein the detergent is OBG (octyl-beta-glucopyranoside) or SDS (Sodium dodecyl sulfate).

22. A method for hydrolyzing a polypeptide at an aspartic acid amino acid residue comprising contacting the polypeptide with a hydrolyzing composition according to claim 1 to obtain polypeptide fragments having aspartic acid residues at the N- or C-terminus and optionally determining the amino acid sequence of resultant polypeptide fragments.

23. A method for hydrolyzing a polypeptide at an aspartic acid amino acid residue comprising contacting the polypeptide with a hydrolyzing composition according to claim 20 to obtain polypeptide fragments having aspartic acid residues at the N- or C-terminus and optionally determining the amino acid sequence of resultant polypeptide fragments.

24. A method of determining the amino acid sequence of a polypeptide comprising:
(i) hydrolyzing the polypeptide with the composition according to claim 1 to obtain polypeptide fragments having aspartic acid residues at the N- or C-terminal ends of the fragments;
(ii) determining the sequence of resultant polypeptide fragments; and
(iii) determining the sequence of the polypeptide by matching and connecting the sequences of the polypeptide fragments so as to obtain the full sequence of the polypeptide.

25. A method of determining the amino acid sequence of a polypeptide comprising:
(i) hydrolyzing the polypeptide with the composition according to claim 20 to obtain polypeptide fragments having aspartic acid residues at the N- or C-terminal ends of the fragments;
(ii) determining the sequence of resultant polypeptide fragments; and
(iii) determining the sequence of the polypeptide by matching and connecting the sequences of the polypeptide fragments so as to obtain the full sequence of the polypeptide.

26. The method according to claim 22, wherein the sequence of polypeptide fragments is determined through mass spectrometry.

27. The method according to claim 26, wherein the water is labeled with deuterium, or tritium, or ¹⁷O or ¹⁸O labeled water.

28. The method according to claim 22, wherein the hydrolysis of the polypeptide is carried out by heating to about 75 to 150° C. reaction temperature.

29. The method according to claim 28, wherein the reaction heat is created by micro wave or ultrasonic wave.

30. The method according to claim 28, wherein hydrolysis of the polypeptide is carried out under a reaction temperature ranging from about 95 to 105° C. in a PCR(Polymerase Chain Reaction) machine.

31. The method according to claim 30, wherein hydrolysis of the polypeptide is carried out by heating bath including the hydrolyzing composition and the polypeptide, and heating lid of the PCR machine at about 95 to 105° C. reaction temperature.

32. The method according to claim 25, wherein the container material for hydrolysis reaction is made of plastic.

33. The method according to claim 32, wherein the plastic is made of polyethylene, polypropylene, high density polyethylene, or low density polyethylene.

34. The method according to claim 25, wherein the method comprises determining the sequence of the polypeptide by a database search using a modified cleavage rule incorporating polypeptide fragments having aspartic acid residues at either the N- or C-terminal ends or both the N- and C-terminal ends.

35. The method according to claim 25, wherein the database search is carried out with a PCA database menu which has a cleavage rule and modification rule incorporating polypeptide fragments having aspartic acid residues at either the N- or C-terminal ends or both the N- and C-terminal ends.

36. A method of determining amino acid sequence of a polypeptide comprising:
(i) hydrolyzing the polypeptide with protease(s) to obtain polypeptide fragments;
(ii) hydrolyzing the composition obtained in step (1) with the composition according to claim 1 to obtain polypeptide fragments having aspartic acid residues at the N- or C-terminal ends of the fragments;
(iii) determining the sequence of resultant polypeptide fragments;
(iv) determining the sequence of the polypeptide by matching and connecting the sequences of the polypeptide fragments so as to obtain the full sequence of the polypeptide.

37. A method of determining amino acid sequence of a polypeptide comprising:
(i) hydrolyzing the polypeptide with the composition according to claim 20 to obtain polypeptide fragments having aspartic acid residues at the N- or C-terminal ends of the fragments;
(ii) hydrolyzing the composition obtained in step (1) with protease(s) to obtain polypeptide fragments;
(iii) determining the sequence of resultant polypeptide fragments;
(iv) determining the sequence of the polypeptide by matching and connecting the sequences of the polypeptide fragments so as to obtain the full sequence of the polypeptide.

38. The method according to claim 36, comprising determining the sequence of the polypeptide by a database search using a modified cleavage rule incorporating polypeptide fragments having aspartic acid residues at either the N- or C-terminal ends or both the N- and C-terminal ends.

39. The method according to claim 38, wherein the database search is carried out with a PCA database menu which has a cleavage rule and modification rule incorporating polypeptide fragments having aspartic acid residues at either the N- or C-terminal ends or both the N- and C-terminal ends.

40. A kit for hydrolyzing polypeptide comprising: (i) a first container containing an acid solution and water; (ii) a second container containing a water miscible organic solvent, wherein the acid solution is at least one selected from the group consisting of trifluoroacetic acid, phosphoric acid, propionic acid, HCl, o-iodobenzoic acid, glacial acetic acid, and any acid having buffering capacity near pH 2.

41. The kit according to claim 40, wherein the second container further contains a reducing agent.

42. The composition according to claim 2, wherein the composition further comprises a detergent.

43. The method according to claim 23, wherein the sequence of polypeptide fragments is determined through mass spectrometry.

44. The method according to claim 24, wherein the sequence of polypeptide fragments is determined through mass spectrometry.

45. The method according to claim 25, wherein the sequence of polypeptide fragments is determined through mass spectrometry.

46. The method according to claim 23, wherein the hydrolysis of the polypeptide is carried out by heating to about 75 to 150° C. reaction temperature.

47. The method according to claim 24, wherein the hydrolysis of the polypeptide is carried out by heating to about 75 to 150° C. reaction temperature.

48. The method according to claim 25, wherein the hydrolysis of the polypeptide is carried out by heating to about 75 to 150° C. reaction temperature.

49. The method according to claim 37, comprising determining the sequence of the polypeptide by a database search using a modified cleavage rule incorporating polypeptide fragments having aspartic acid residues at either the N- or C-terminal ends or both the N- and C-terminal ends.
